(19) United States
(12) Patent Application Publication
(10) Pub. No.: US 2010/0041079 A1
(43) Pub. Date: Feb. 18, 2010

(54) METHOD FOR PREDICTING CARDIOVASCULAR EVENTS

(75) Inventors: Salim Yusuf, Burlington (CA); Jack Hirsh, Burlington (CA); John Eikelboom, Ancaster (CA)

Correspondence Address: OCCHIUTI ROHLICEK & TSAO, LLP, 10 FAWCETT STREET, CAMBRIDGE, MA, 02138, US

(73) Assignee: McMaster University, Hamilton (CA)

(21) Appl. No.: 12/543,355

(22) Filed: Aug. 18, 2009

Related U.S. Application Data
(63) Continuation-in-part of application No. 10/670,118, filed on Sep. 24, 2003, now Pat. No. 7,595,201.
Application No. 10/670,118 is a continuation-in-part of application No. PCT/CA03/00422, filed on Mar. 24, 2003.
(60) Provisional application No. 60/367,883, filed on Mar. 24, 2002.

(51) Int. Cl. G01N 33/53

(52) U.S. Cl. 435/7.92

(57) ABSTRACT

A novel method for assessing the risk of a cardiovascular event is provided. The concentration of 11-dehydro thromboxane in a urine sample is measured and compared to a set of standardized quartile concentrations. A concentration of urinary 11-dehydro thromboxane that falls within the fourth quartile is indicative of a greatly increased risk of a recurrent cardiovascular event.

### CROSS-REFERENCE TO RELATED APPLICATION PARAGRAPH

This application is a Continuation-in-Part and claims the benefit of U.S. Utility application Ser. No. 10/670,118 filed on Sep. 24, 2003, which is a Continuation-in-Part of PCT/CA03/00422 filed on Mar. 24, 2003 which claims priority to U.S. Provisional Application No. 60/367,883 filed on Mar. 24, 2002, the contents of which are hereby incorporated by reference in their entirety.

### FIELD OF THE INVENTION

The present invention relates to the rapid detection of aspirin resistance as an indicator of the risk of cardiovascular events. Particularly the invention relates to methods and devices for the measurement of suppression of thromboxane generation in response to treatment with aspirin.

### BACKGROUND OF THE INVENTION

Cardiovascular disease ranks as a leading cause of mortality and morbidity and represents a significant drain on health resources in many countries.

It is well established that aspirin therapy reduces the risk of a stroke and a first heart attack in healthy individuals, and subsequent heart attacks, strokes, or cardiovascular death in patients with established cardiovascular disease. For example, U.S. Pat. No. 5,240,917 relates to the percutaneous administration of aspirin as an antithrombotic agent.

Studies have shown that aspirin reduces the risk of cardiovascular events by as much as 25% in patients with arterial vascular disease.

Most heart attacks and strokes are caused by blood clots in the heart or brain arteries that form on top of cracked atherosclerotic plaques. These blood clots are predominantly composed of clumped platelets. Aspirin works to prevent blood clot formation at these sites by reducing the ability of the platelets to clump together and form platelet aggregates. Aspirin, also known as acetylsalicylic acid, reduces platelet reactivity because its acetyl group acetylates a key intra-platelet enzyme known as cyclo-oxygenase. Once acetylated, cyclo-oxygenase cannot work to generate thromboxane A2, a substance released from the platelets that serves to activate other platelets and induce them to clump together in aggregates. In order for aspirin to work, therefore, it must reduce thromboxane A2 levels.

Thromboxane A2 has a very short half-life, and is rapidly converted to a stable metabolite called thromboxane B2. Although thromboxane B2 can be measured in blood, the tests can be problematic because platelets can be activated during the collection process. Once activated, the platelets will release thromboxanes that can interfere with the assay. It is therefore preferable to measure thromboxane B2 in the urine.

Even though platelets are an important part of blood clots, rapid technology to measure and predict platelet physiology is lacking. Some accepted laboratory methods include:
i) Bleeding Time, a test which is qualitative, not quantitative;
ii) Platelet Aggregometry. This test measures the clumping of platelets in response to various stimuli. The test is arduous, time-consuming, and expensive and is not specific for the effects of aspirin on platelet activation.
iii) Tests of platelet activation using fluorescent cell sorting techniques. This test can only be done on freshly collected blood and uses size separation to separate platelets from other blood cells and fluorescently-tagged antibodies to identify activated platelets. This test is cumbersome and does not provide aspirin-specific information.

The present invention provides a novel method for assessing platelet function and correlating a readout of that function with the risk of a cardiovascular event.

Aspirin is effective for patients with heart attacks, strokes or peripheral arterial disease or those at risk of these disorders. Aspirin has also been shown to be effective in reducing the incidence of pregnancy-induced hypertension and pre-eclamptic toxicity in women at risk. A role for aspirin in reducing the risk of fatal colon cancer has also been suggested and aspirin may be useful in the treatment of patients with antiphospholipid antibodies, including the lupus anticoagulant. Thus, determining the effectiveness of aspirin treatment in many conditions is an important prognostic factor and may help physicians recommend the most appropriate therapeutic course.

While aspirin is effective in many individuals, approximately 10 to 20% of patients with arterial thrombosis who are treated with aspirin have a recurrent vascular event during long-term follow-up. The failure of these patients to derive a beneficial effect from aspirin is termed “aspirin resistance”. There are several possible explanations for aspirin resistance but, whatever the underlying cause, the result is the same. It would obviously be beneficial to be able to identify those patients who are aspirin resistant in order to help physicians determine the advisability of altering the aspirin dose or administering alternative or additional anti-platelet therapies. A need therefore exists for a simple method to accurately determine the response to aspirin and predict the likelihood of onset of a cardiovascular event or other medical condition that would benefit from lowering of thromboxane-A2 levels.

### SUMMARY OF THE INVENTION

To be able to identify those people at particular risk of having a recurrent vascular event, so that they can be appropriately treated before a heart attack or stroke occurs, would be of great clinical importance. Former attempts to develop predictive assays, particularly those utilizing blood, have had mixed results. Thus, it is an object of one aspect of the present invention to provide a rapid, non-invasive, reproducible method for determining aspirin resistance. The present invention demonstrates for the first time an association between aspirin resistance, defined as failure of suppression of thromboxane generation, and cardiovascular risk. Determination of the degree of resistance to aspirin is used to predict the risk of a cardiovascular event or other condition that would benefit from lowering thromboxane A2 levels.

The present invention is based on the observation that urinary thromboxane A2 metabolite levels in patients are a surprisingly accurate predictor of recurrent cardiovascular mortality. Thus, determination of metabolite levels in patients may serve to identify those patients at particular risk of developing cardiac ischemia or stroke.

In one aspect of the invention, a method for assessing a relative risk of a cardiovascular event in a patient is provided. The method comprises the comparison of a concentration of thromboxane B2, preferably 11-dehydrothromboxane B2, in a body fluid of the patient to a set of standardized concentrations wherein the comparison is indicative of a relative risk for a cardiovascular event in the patient.

The set of standardized thromboxane, preferably 11-dehydrothromboxane B2, concentrations is a set of concentration-based quartiles.

The bodily fluid may be blood, plasma, serum or urine. Urine is the preferred bodily fluid.

The concentration of 11-dehydrothromboxane B2 in the bodily fluid is typically determined using an immunoassay. A preferred immunoassay is an ELISA. The immunoassay is preferably a competitive binding assay based on a determination of the amount of 11-dehydrothromboxane B2 in the body fluid compared to a known quantity of labeled 11-dehydrothromboxane B2 able to bind to an immobilized anti-11-dehydrothromboxane B2 antibody.

A method for assessing aspirin resistance in a patient is provided. The method comprises determining the concentration of a metabolite of thromboxane A2 in a sample of body fluid from the patient. The method preferably further comprises the step of comparing the concentration of metabolite in the sample to a predetermined set of concentration quartiles to determine within which quartile the sample falls and determining aspirin resistance based on the quartile of the sample. A concentration of metabolite within the second, third or fourth quartile is indicative of an increased risk of a cardiovascular event.

In another aspect, a method for assessing the risk of a cardiovascular event in a patient is provided. The method comprises determining the level of thromboxane B2 or another thromboxane A2 metabolite in a body fluid, preferably urine. In a preferred embodiment, the method comprises an immunoassay in which a body fluid sample from the patient is contacted with an antibody that specifically binds to a metabolite of thromboxane-A2. The formation of immune complexes is then detected to determine the level of antigen in the sample and the sample level thus obtained is compared to control levels to determine a relative risk factor.

In another aspect, there is provided a method of screening a patient for risk of having a cardiovascular event which comprises contacting a body fluid sample from the patient with an antibody which specifically binds to a thromboxane-A2 metabolite, determining the degree of immune complex formation by immunoassay, and assessing the patient's risk of a cardiovascular event upon the basis of immune complex formation.

In a preferred embodiment, the patient has arterial vascular disease and the method is used to predict the risk of a recurrent vascular event.

In a further preferred embodiment, the metabolite that is measured is thromboxane-B2 metabolite, preferably 11-dehydro thromboxane B2.

In a further aspect, a urine level of this metabolite of greater than 15 ng/mmol creatinine is indicative of risk of a cardiovascular event, more preferably a urine level greater than 21.9 ng/mmol creatinine is indicative of risk of a cardiovascular event and most preferably a urine level greater than 33.8 ng/mmol creatinine is indicative of risk of a cardiovascular event.

The present invention also provides a kit for assessing aspirin resistance. The kit typically comprises (a) an antibody that specifically binds to a thromboxane A2 metabolite, and (b) a labeled sample of the metabolite.

In another aspect of the invention, a device for detecting 11-dehydro thromboxane B2 in a test sample obtained from a mammal is provided. The device comprises an immobilized moiety that specifically binds to 11-dehydro thromboxane B2 and means for visually determining if the level of 11-dehydro thromboxane exceeds a predetermined threshold amount. The moiety that specifically binds 11-dehydro thromboxane B2 is preferably an antibody, an antibody fragment, a single chain antibody or an antigen-binding domain of an antibody. The binding moiety is immobilized on a solid support selected from the group consisting of glass, polystyrene, nylon, cellulose acetate, nitrocellulose and other polymers. The device may be in the format of a dipstick.

In yet another aspect of the invention a method of predicting increased risk of an increased risk for a recurrent cardiovascular event is provided. The method comprises:
a) measuring the concentration of 11-dehydro thromboxane B2 in a test urine sample;
b) comparing the concentration of the test sample to the quartile concentration of a series of reference samples;
c) determining which quartile concentration the test sample falls within; and
d) predicting the risk based on the corresponding quartile concentration.

In a particularly preferred embodiment, an immunoassay device for detecting the presence of an analyte is provided. The device comprises a strip that comprises a reagent that specifically binds to the analyte to be tested. The reagent is preferably distributed in patches correlating to quartiles to detect different amounts of the analyte.

The methods and devices of the present invention can prospectively identify patients who are relatively resistant to anti-thrombotic doses of aspirin and who may benefit from higher doses of aspirin or additional or alternative therapies that can either block thromboxane production or activity or inhibit another pathway of platelet activation.

### BRIEF DESCRIPTION OF THE DRAWINGS

FIG. 1 demonstrates graphically the relationship between quartiles of 11-dehydro thromboxane B2 levels and risk of a cardiovascular event; and

FIG. 2 illustrates one embodiment of a test device for measurement of thromboxane B2 levels.

### DETAILED DESCRIPTION OF THE INVENTION

Survivors of acute myocardial infarction are at greatly increased risk for subsequent fatal and non-fatal cardiovascular events. This heightened risk is influenced by many factors, such as age, co-morbid diseases and response to treatment.

The term “cardiovascular event(s) as used herein refers to coronary and/or cerebrovascular event(s) including primary myocardial infarction, secondary myocardial infarction, angina pectoris (including unstable angina), congestive heart failure, sudden cardiac death, cerebral infarction, syncope, transient ischemic attack and the like.

While aspirin has been known to reduce thromboxane-A2 levels, the present invention provides a novel method for determining aspirin resistance. The degree of aspirin resistance can be used to predict the occurrence of a cardiovascular event based on the surprising result that there is a correlation between the quartile in which a level of 11-dehydro thromboxane B2 falls and the incidence of myocardial infarction, stroke and cardiovascular death.

The present invention provides a method for determining the risk of a recurrent cardiovascular event based on the level of thromboxane-A2 produced in response to treatment with aspirin. The level of thromboxane-A2 generation is determined by measuring urinary levels of metabolites of thromboxane-A2. A preferred stable metabolite of thromboxane-A2 which can be measured is thromboxane B2, more particularly, 11-dehydro thromboxane B2.

In responsive individuals, aspirin reduces levels of thromboxane A2 by irreversibly acetylating the enzyme, cyclo-oxygenase 1. However, a subpopulation of individuals does not exhibit this inhibition of thromboxane generation in response to aspirin. The incomplete suppression of thromboxane generation with the usual dose (75 to 325 mg/d) of aspirin is termed aspirin resistance. In patients with cardiovascular disease taking aspirin, those who are aspirin resistant are more likely to have a recurrence of a cardiovascular event. The present invention provides a method for identifying those patients who are aspirin resistant. In addition to the prediction of a cardiovascular event, aspirin resistance may be an important factor in the selection of a treatment for other conditions that would benefit from a lowering of thromboxane levels.

The present invention provides a method for establishing quartiles of thromboxane B2 levels in response to administration of aspirin and correlating those quartile levels with risk of a cardiovascular event.

The utility of the method was demonstrated in a clinical trial. The levels of thromboxane B2 were determined for a large number of patients. A range of thromboxane B2 levels was established and the range was then divided into four quartiles. Patients were followed up to determine the occurrence and therefore relative risk of a subsequent event for a value falling within a particular quartile.

The initial study was an international, randomised, placebo-controlled, two-by-two factorial trial of ramipril and vitamin E for the secondary prevention of cardiovascular disease. The institutional review committee at each participating center approved the study and all subjects gave informed consent.

A total of 9,541 patients aged at least 55 years at the time of randomization who had a history of coronary artery disease, stroke, peripheral vascular disease, or diabetes plus at least one other cardiovascular risk factor were assigned to one of four treatments: ramipril titrated up to 10 mg daily, vitamin E 400 IU daily, both, or neither.

All study participants were asked to provide a first morning urine specimen at the time of randomisation. Of the 9,541 patients in the study, 9,282 (97%) provided baseline urine samples. Samples (n=5529) from the 129 Canadian centres participating in this study were sent to the central laboratory in Hamilton, Canada where they were stored at −800 C until analysis. Only samples from Canadian centres were used for the present study.

All patients in the study were followed at one month, six months, and six monthly intervals thereafter until completion of the study. At each follow-up, clinical outcomes were recorded and medication use, including aspirin, was documented. The primary outcome was the composite of myocardial infarction, stroke, and death from cardiovascular causes.

Of patients with available urine samples (n=5529), only those who were taking aspirin at the time of commencement of the run-in phase (prior to randomisation), at randomisation (coinciding with the time of urine collection), and at each follow-up visit, were eligible for inclusion. Aspirin-treated patients who provided an adequate baseline sample of urine and had a confirmed myocardial infarction, stroke, or cardiovascular death after randomisation were defined as cases. Controls were randomly selected from aspirin-treated patients who provided an adequate baseline urine sample but did not experience myocardial infarction, stroke, or cardiovascular death after randomisation. Controls were matched according to gender and age (±5 years) in a 1:1 ratio with cases.

For each case and control, urine collected and stored at baseline was thawed and assayed for 11-dehydro thromboxane B2 levels using a commercially available enzyme immunoassay (Cayman Chemical, Ann Arbor, Mich.) that has inter- and intra-assay coefficients of variation of 12.1% and 10%, respectively. Assays were performed by laboratory staff blinded to patient status as case or control. In addition, case and control specimens were assayed in random order, thereby reducing the possibility of systematic bias.

Means or proportions for baseline demographics and risk factors were calculated for cases and controls. The significance of any difference between cases and controls was tested using Student's paired t-test for means and McNemar chi square test for proportions, which takes into account the matching between cases and controls. Because 11-dehydro thromboxane B2 values are skewed, geometric means were calculated after log transformation of the raw data and the significance of any differences in geometric mean values between cases and controls was tested using Student's paired t-test. Median concentrations also were calculated and levels in cases and controls were compared using Wilcoxon's rank-sum test.

Tests for trend were used to assess any association between increasing baseline urinary 11-dehydro thromboxane B2 concentrations and risk of myocardial infarction, stroke, or cardiovascular death after dividing the samples into quartiles defined by the distribution of the complete cohort. Adjusted estimates of the association between increasing baseline urinary 11-dehydro thromboxane B2 concentrations and risk of myocardial infarction, stroke, or cardiovascular death were obtained using conditional logistic regression modelling that accounted for the matching variables and controlled for the random treatment assignment and baseline differences between cases and controls. A separate multivariable regression model was used to examine the association between baseline patient characteristics, including age, gender, heart rate, blood pressure, body mass index, past history of vascular disease, conventional vascular risk factors, lipid-lowering therapy, beta-blockers, diuretics, and randomised treatment allocation (ramipril or vitamin E), and urinary 11-deydro thromboxane B2 concentrations in the urine.

All P-values are two-sided and confidence intervals are calculated at the 95 percent level.

Baseline characteristics of cases and controls are shown in Table 1. As expected, patients in whom myocardial infarction, stroke, or cardiovascular death subsequently developed had a higher mean body mass index and baseline blood pressure and were more likely than those who remained free of these events to be current smokers or have a history of hypertension, diabetes, myocardial infarction, or peripheral vascular disease. Cases also were more often treated with diuretics or calcium channel blockers at baseline and less often treated with lipid-lowering drugs or randomised to ramipril therapy. Because of the matching, the age and gender of cases and controls were similar.

TABLE 1
Baseline characteristics of study participants.*

| Characteristic† | Cases (n = 488) | Controls (n = 488) | P-value |
|---|---|---|---|
| Age - yr | 67.3 ± 7.2 | 67.4 ± 7.2 | 0.78 |
| Female sex - no. (%) | 77 (15.8) | 77 (15.8) | — |
| Body mass index‡ | 27.8 ± 4.1 | 26.9 ± 3.7 | <0.001 |
| Heart rate - beats/min | 66.2 ± 10.3 | 65.6 ± 10.9 | 0.41 |
| SBP - mm Hg | 137.1 ± 20.6 | 133.5 ± 18.0 | 0.002 |
| DBP - mm Hg | 76.6 ± 9.8 | 75.6 ± 9.4 | 0.08 |
| History of coronary disease - no. (%) | | | |
| Any | 469 (96.1) | 464 (95.1) | 0.54 |
| MI | 364 (74.6) | 309 (63.4) | <0.001 |
| Stable angina | 355 (72.7) | 336 (68.9) | 0.19 |
| Unstable angina | 184 (37.7) | 176 (36.1) | 0.65 |
| CABG | 176 (36.1) | 154 (31.6) | 0.15 |
| PCI | 87 (17.8) | 104 (21.3) | 0.22 |
| Stroke or TIA - no. (%) | 59 (12.1) | 40 (8.2) | 0.06 |
| Peripheral vascular disease - no. (%) | 240 (49.2) | 173 (35.5) | <0.001 |
| Hypertension - no. (%) | 219 (44.9) | 154 (31.6) | <0.001 |
| Diabetes - no. (%) | 159 (32.6) | 105 (21.5) | <0.001 |
| Elevated total cholesterol - no. (%) | 279 (57.2) | 310 (63.5) | 0.38 |
| Current cigarette smoking - no. (%) | 81 (16.6) | 57 (11.7) | 0.03 |
| Medications - no. (%) | | | |
| Aspirin | 488 (100) | 488 (100) | — |
| β-blocker | 241 (49.4) | 235 (48.2) | 0.76 |
| Lipid-lowering agent | 121 (24.8) | 166 (34.0) | 0.002 |
| Diuretics | 73 (15.0) | 34 (7.0) | <0.001 |
| Calcium channel blockers | 289 (59.2) | 238 8.8) | 0.002 |
| Ramipril | 227 (46.5) | 274 (56.1) | 0.003 |
| Vitamin E | 246 (50.4) | 252 (51.6) | 0.74 |

*Plus-minus values are mean ± SD.
†CABG denotes coronary artery bypass graft surgery, CV cardiovascular, DBP diastolic blood pressure, MI myocardial infarction, PCI percutaneous coronary intervention, SBP systolic blood pressure, TIA transient ischaemic attack.
‡The body-mass index is the weight in kilograms divided by the square of the height in meters.

Geometric mean and median urinary concentrations of 11-dehydro thromboxane B2 at baseline were significantly higher among patients who subsequently developed the composite outcome of myocardial infarction, stroke, or cardiovascular death compared with those who remained free of these events (Table 2). The difference between cases and controls was greatest in those who suffered a myocardial infarct (24.5 vs. 20.9 ng/mmol creatinine, P=0.003) or died from a cardiovascular cause (25.6 vs. 20.4 ng/mmol creatinine, P<0.001).

TABLE 2
Baseline urinary concentrations of urinary 11-dehydro
thromboxane B2 in cases and controls.

| | 11-dehydro | | |
|---|---|---|---|
| | thromboxane B2 | | |
| | concentration | | |
| | (ng/mmol creatinine) | | |
| Outcome | Cases | Controls | P-value |
| MI, Stroke or CV death (n = 488) | | | |
| Geometric mean | 24.5 | 21.5 | 0.01 |
| Median | 22.7 | 21.0 | 0.01 |
| MI (n = 378) | | | |
| Geometric mean | 24.5 | 20.9 | 0.003 |
| Median | 22.8 | 20.3 | 0.001 |
| Stroke (n = 80) | | | |
| Geometric mean | 25.0 | 27.4 | 0.47 |
| Median | 21.3 | 25.9 | 0.40 |
| CV death (n = 244) | | | |
| Geometric mean | 25.6 | 20.4 | <0.001 |
| Median | 24.0 | 19.9 | <0.001 |

*MI denotes myocardial infarction, CV cardiovascular.

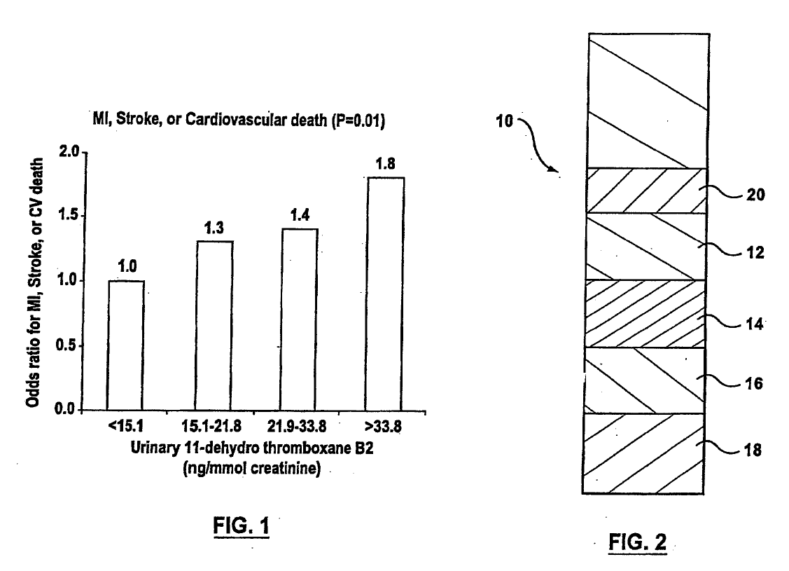

The adjusted odds for the composite outcome of myocardial infarction, stroke, or cardiovascular death increased with each increasing quartile of baseline urinary 11-dehydro thromboxane B2 concentration (P for trend across quartiles, 0.01), with patients in the highest quartile having a risk 1.8-fold higher than those in the lowest quartile (Odds Ratio [OR] 1.8; 95 percent confidence interval [CI] 1.2-2.9, P=0.009) (FIG. 1). A similar association was seen with myocardial infarction (P for trend across quartiles, 0.005) and cardiovascular death (P for trend across quartiles, 0.001) (Table 3). Results were similar with or without adjustment for baseline differences between cases and controls including conventional vascular risk factors, co-interventions, and randomised treatment allocation.

TABLE 3
Adjusted odds* of future cardiovascular death, myocardial infarction, and stroke
according to baseline urinary concentrations of 11-dehydro thromboxane B2.

| | Quartiles of 11-dehydro thromboxane B2 | | | | |
|---|---|---|---|---|---|
| | concentration (ng/mmol creatinine) | | | | |
| Outcome† | <15.1 | 15.1-21.8 | 21.9-33.7 | >33.7 | trend P |
| MI/Stroke/CV death (n = 488) | | | | | |
| Odds ratio (95 CI) | 1.0 | 1.3 (0.9-2.0) | 1.4 (0.9-2.2) | 1.8 (1.2-2.7) | 0.01 |
| P-value | — | 0.13 | 0.09 | 0.009 | |
| MI (n = 378) | | | | | |
| Odds ratio (95 CI) | 1.0 | 1.3 (0.8-2.1) | 1.5 (1.0-2.5) | 2.0 (1.2-3.4) | 0.005 |
| P-value | — | 0.26 | 0.07 | 0.006 | |
| Stroke (n = 80) | | | | | |
| Odds ratio (95 CI) | 1.0 | 2.5 (0.6-10.0) | 0.6 (0.2-2.2) | 0.6 (0.2-1.8) | 0.20 |
| P-value | — | 0.18 | 0.45 | 0.34 | |
| CV death (n = 244) | | | | | |
| Odds ratio (95 CI) | 1.0 | 2.0 (1.0-3.9) | 2.5 (1.3-4.9) | 3.5 (1.7-7.4) | 0.001 |
| P-value | — | 0.06 | 0.006 | <0.001 | |

*Adjusted for baseline differences between cases and controls.
†CI denotes confidence interval, MI myocardial infarction, CV cardiovascular.

To evaluate whether increased baseline urinary 11-dehydro thromboxane B2 concentrations were associated with early rather than late cardiovascular events separate analyses were performed in patients who experienced an event within the first 12 months of study entry and those whose event occurred more than 12 months after study entry. The adjusted odds for the composite outcome of myocardial infarction, stroke, or cardiovascular death that was associated with the highest quartile of urinary 11-dehydro thromboxane B2 as compared with the lowest quartile was 2.9 (95% CI: 0.9-9.1) for events occurring with the first 12 months and 1.7 (95% CI: 1.0-2.7) for events occurring after the first 12 months.

Using linear multivariable regression modeling, variables that were found to be independently associated with baseline urinary 11-dehydro thromboxane B2 concentrations in the urine were: female gender (P=0.004); body mass index (P=0.001), history of peripheral vascular disease (P=0.01), current cigarette smoking (P=0.09), use of calcium channel blockers (P=0.08), and randomisation to vitamin E (P=0.04). However, these variables combined were able to predict less than 5% of the variation in urinary 11-dehydro thromboxane B2 concentrations (R-square 0.045).

FIG. 1 illustrates the association between quartiles of 11-dehydro thromboxane B2 levels and composites of myocardial infarction (MI), stroke, or cardiovascular (CV) death that was seen in an exemplary study. The results indicate that if the test value falls within the first quartile, there is an absolute risk of approximately 10%. If the test value falls within the second quartile, there is an absolute risk of about 13% over 5 years. If the test value fall within the third quartile, the absolute risk is about 14% and if the value falls within the fourth quartile, the risk is about 18%.

These results indicate that urinary thromboxane B2 levels can be used an indicator of aspirin resistance and that aspirin resistance is a valuable predictor of the occurrence of a cardiovascular event.

The method for assessing the risk of a cardiovascular event according to the present invention comprises measuring thromboxane B2 levels, as an indicator of aspirin resistance, in a urine sample, comparing the level to a set of predetermined quartiles and determining into which quartile the sample level falls. The association of a test level within a quartile range is indicative of the long-term relative risk of myocardial infarction, stroke and vascular death. The risk is progressive according to the quartile. Sample levels in the first quartile are associated with a low level of risk and samples in the fourth quartile are indicative of a very high level of risk. The level of TxB2 in urine can be determined in a variety of ways. A few types of assays that can be used are ELISA, EIA, RIA, FIA, dip stick, flow through, etc. and these can be performed in different ways. For example, a competitive immunoassay (either sequential or concurrent), a sandwich immunoassay, a direct immunoassay or other type of immunoassay can be used.

In a competitive immunoassay, a solid support, usually, but not always, a microtiter plate is coated with an anti mouse 1gG antibody. This can be goat, rat, rabbit, chicken, sheep antibodies, monoclonal or polyclonal, that bind mouse IgG. A mouse IgG monoclonal antibody specific for TxB2 is prepared. The competitive assay can be performed in a sequential or simultaneous manner. A control standard curve is determined. TxB2 is labelled with a reporter molecule. The reporter molecule could be an enzyme, a radioclide, a fluorophore or a luminescent protein.

In a sequential competitive assay, the sample is incubated with the a TxB2 antibody. The antibody will be captured by the anti-mouse immunoglobulin and many of the sites will be occupied. The labelled TxB2 is then added. Depending on how much TxB2 is already bound to the surface, there is more or less room for the labelled (tracer) TxB2 to bind. The level of signal from the tracer is inversely related to the amount of TxB2 in the sample.

In a simultaneous competitive assay, the labelled and unlabelled TxB2 are added simultaneously and compete with each other for binding to the mouse a TxB2 antibody.

A typical protocol for a competitive assay is as follows:
a) Unlabeled antibody is incubated in the presence of its labelled antigen.
b) These bound antibody/antigen complexes are then added to an antibody coated well. The plate is washed and the sample is added, so that unbound antibody is removed.
c) Alternatively the unlabelled antibody can be immobilized on the bottom of the well either directly or via an antibody specific for it and labelled antigen and the sample are added.
d) A detecting antibody, coupled to an enzyme is added.
e) A substrate is added, and remaining enzymes elicit a chromogenic or fluorescent signal.
   In a competitive assay, the higher the antigen concentration in the sample, the weaker the signal as the antigen in the sample will compete with the labelled antigen for binding to the specific antibody.

In a sandwich assay, the plate is coated with an antibody specific for TxB2. The sample is added to the plate. An antibody specific for TxB2 is then added. Then a labelled antibody specific for the type of the bound antigen (e.g. mouse) is then added. The amount of label/signal produced is indicative of the amount of TxB2 in the sample. Of course, a standard curve is first established.

A standard protocol for a sandwich assay is as follows:
a) Prepare a surface to which a known quantity of capture antibody is bound.
b) Block any non specific binding sites on the surface.
c) Apply the antigen-containing sample to the plate.
d) Wash the plate, so that unbound antigen is removed.
e) Apply primary antibodies that bind specifically to the antigen.
f) Apply enzyme-linked secondary antibodies which are specific to the primary antibodies.
g) Wash the plate, so that the unbound antibody-enzyme conjugates are removed.
h) Apply a chemical which is converted by the enzyme into a color or fluorescent or electrochemical signal.
i) Measure the absorbency or fluorescence or electrochemical signal (e.g., current) of the plate wells to determine the presence and quantity of antigen.

The concentration of thromboxane B2, particularly 11 dihydrothromboxane can also be detected using a radioimmunoassay. Radioimmunoassay (RIA) is a very sensitive technique used to measure concentrations of antigens (for example, levels in the blood or urine).

The RIA technique is extremely sensitive and extremely specific. However, this assay requires special precautions, since radioactive substances are used.

To perform a radioimmunoassay, a known quantity of an antigen is made radioactive, frequently by labeling it with gamma-radioactive isotopes of iodine attached to tyrosine. This radiolabeled antigen is then mixed with a known amount of antibody for that antigen, and as a result, the two chemically bind to one another. Then, a sample of serum or urine from a patient containing an unknown quantity of that same antigen is added. This causes the unlabeled (or “cold”) antigen from the serum to compete with the radiolabeled antigen for antibody binding sites. As the concentration of “cold” antigen is increased, more of it binds to the antibody, displacing the radiolabeled variant, and reducing the ratio of antibody-bound radiolabeled antigen to free radiolabeled antigen. The bound antigens are then separated from the unbound ones, and the radioactivity of the free antigen remaining in the supernatant is measured. Using known standards, a binding curve can then be generated which allows the amount of antigen in the patient's serum to be derived.

The term “antibody” is used herein to refer to a monoclonal or polyclonal antibody or an antibody fragment having specific binding affinity. The term “antibody fragment” refers to a portion of an antibody, such as an antigen binding domain, a hypervariable domain of either the heavy or light chain and the term also includes single chain antibodies.

Some examples of solid supports that can be used in the present invention include microtiter plates, tubes, polystyrene beads, nylon, nitrocellulose, cellulose acetate, glass fibers and other types of porous polymers.

The immunoassay determination of thromboxane A2 metabolites can be performed using monoclonal or polyclonal antibodies, which may be raised using techniques conventional in the art. For example, antibodies may be made by injecting a host animal, e.g. a mouse or rabbit, with the antigen. The antigen may be conjugated with an immunogenic protein such as PPD, a protein derivative of tuberculin, Keyhole Limpet Haemocyanin, BSA etc., to provide either a serum containing polyclonal antibodies or spleen cells for fusion to provide hybridomas or immortalised cell lines. Other standard methods may also be used.

Suitable labels for antibodies or antigens include radionucleides, fluorophores, chemiluminescent labels, bioluminescent labels, enzymes, for example as used in ELISA systems, dyes or particles such as colloidal gold.

It is clearly apparent that different methods can be used to measure thromboxane B2 levels in urine. The absolute values corresponding to aspirin resistance varies depending on the diagnostic method used. In addition, different readouts can be used to report levels of thromboxane B2. For example, thromboxane B2 concentration can be reported as ng/mmol creatinine, pg/mg of creatinine, μg/L, pg/ml and so on.

The invention is not limited to a particular assay or an absolute readout value. The ranges of each of the quartiles will vary depending on the assay used and the read-out value that is used. Regardless of the type of assay used or the readout used, the method comprises dividing a range of thromboxane B2, preferably 11 dihydrothromboxane B2, into quartiles and then determining into which quartile a sample concentration falls, wherein the relative risk increases from quartile 1 through to quartile 4.

The method for determining aspirin resistance and relative risk of a cardiovascular event comprises:
a) contacting a body fluid with an antibody reactive with 11-dehydro thromboxane for a time and under conditions sufficient to form an antigen-antibody complex and detecting the antigen-antibody complex formed;
b) quantitating the amount of complex formed in step a);
c) comparing the amount of complex quantitated in step b) with standardized quartile concentrations, and
d) determining into which quartile the sample concentration falls,
   wherein a higher quartile of 11-dehydro thromboxane concentration correlates with aspirin resistance and with the risk of a subsequent cardiovascular event.

The invention also provides a method of screening patients, for risk of having a cardiovascular event or other condition which would benefit from the reduction of thromboxane A2 levels. A body fluid of the patient is tested for the concentration of a thromboxane A2 metabolite or a fragment thereof and an assessment of the patient's risk is made upon the basis of comparing the level to a set of concentration quartiles.

Such screening may be positive i.e. to identify those patients at risk and consequently in need of alternative treatment or negative to eliminate those patients who are not at significant risk from extensive follow-up.

The body fluid on which the determination is performed may be any body fluid in which 11 dehydro thromboxane may be located. It is preferably urine. In some cases it may be convenient to extract the peptide, or otherwise treat the sample prior to determination.

11-dehydro thromboxane B2 is highly stable on storage and therefore a reliable prognosis may be obtained when the determination is performed on samples that have been stored for some time. This is advantageous in that it facilitates assay reproducibility and it enables the assay to be delayed post sample collection. Another advantage of the method of the invention is that the 11 dehydro thromboxane B2 determination can be performed with high specificity and sensitivity leading to an accurate and reliable prediction of recurrent cardiovascular events. Prior art methods do not approach this level of accuracy and sensitivity.

In another aspect of the invention, the components needed to perform the immunoassay are supplied in kit form. Such a kit would comprise:
(a) an antibody capable of binding to 11-dehydro thromboxane A2, said antibody in an immobilised form;
(b) a control preparation of 11 dehydro thromboxane B2; and
(c) a labelled secondary antibody specific for 11 dehydro thromboxane B2.

One preferred embodiment of a test kit for the determination of 11-dehydro thromboxane levels is shown in FIG. 2. A test strip 10 is provided which is divided into test patches, 12, 14, 16 and 18. Each patch has a reagent that reacts to the presence of 11-dehydro thromboxane B2, and each reagent is applied so it will react with a predetermined concentration of 11-dehydro thromboxane B2, in a given amount of time. In a particularly preferred embodiment, each of the patches, 12, 14, 16 and 18 are adjusted to react to a quartile concentration of 11-dehydro thromboxane B2 in urine. A series of quartiles is predetermined and the relative risk of a cardiovascular event increases with each quartile. Each patch is processed so a dye or other calorimetric agent provides a readout of the level 11-dehydro thromboxane B2 present in the urine. The reagents, antibodies and other assaying and indicating means, as well as methods of processing are well known to the art. The test strip 10 may also include a patch 20 that changes color when the patch has been in the urine an appropriate amount of time to obtain the desired reaction. The reagents used for this patch 20 would react to substances in the urine which are well known to the art. In some preferred embodiments the patches 12, 14, 16, 18 also have printed on each of them the absolute and relative risk factors associated with the amount of 11-dehydro thromboxane B2 detected on that particular patch. This would allow the clinician to directly obtain the risk to the patient tested from the test strip itself. This would speed diagnosis and avoid errors.

While the present invention has been described in conjunction with specific reference to thrombaoxane B2, the measurement of analyte or simultaneous measurement of two or more analytes (such as 11-dehydro thromboxane B₂ and creatinine) can also be performed using existing rapid testing technologies such as, but not limited to biosensors or membrane based dipstick, lateral flow or chromatographic strips.

Other thromboxane A2 metabolites can be measured as an indicator of aspirin resistance. It is apparent that for the detection of other metabolites, other antibodies that have an affinity for those metabolites will be substituted for the purpose of analyzing the presence and amount of these other proteins.

The correlation of the degree of aspirin resistance with relative risk is an important indicator for improved long-term overall survival and reduced mortality and morbidity due to major cardiovascular events. In particular, 11-dehydro thromboxane levels are divided in quartiles and the quartile in which a particular sample falls is predictive of the occurrence of a cardiovascular event. By recognizing aspirin resistance and its implications, overall deaths can be reduced and congestive heart failure requiring hospitalization can be reduced. The detection of aspirin resistance is also important for the development of an appropriate treatment strategy for other condition which may benefit from a reduction thromboxane A2 levels.

Changes in form and substitution of equivalents are contemplated as circumstances may suggest or render expedient. Although specific terms have been employed herein, such terms are intended in a descriptive sense and not for purposes of limitation.

### REFERENCES

1. Eikelboom J. et al. Aspirin resistance and the risk of myocardial infarction, stroke, or cardiovascular death in patients at high risk for cardiovascular events. Circulation 2002; 105:1650

I claim:

1. A method for assessing a relative risk of a cardiovascular event in a patient comprising the comparison of a concentration of 11-dehydrothromboxane B2 in a body fluid of the patient to a set of standardized 11-dehydrothromboxane B2 concentrations wherein the comparison is indicative of a relative risk for a cardiovascular event in the patient.

2. The method of claim 1 wherein the set of standardized 11-dehydrothromboxane B2 concentrations is a set of concentration-based quartiles.

3. The method of claim 1 wherein the bodily fluid is urine.

4. The method of claim 1 wherein the bodily fluid is blood.

5. The method of claim 1 wherein the concentration of 11-dehydrothromboxane B2 in the bodily fluid is determined using an immunoassay.

6. The method of claim 5 wherein the immunoassay is an ELISA.

7. The method of claim 6 wherein the immunoassay is a competitive binding assay, said competitive binding assay based on a determination of the amount of 11-dehydrothromboxane B2 in the body fluid compared to a known quantity of labeled 11-dehydrothromboxane B2 able to bind to an immobilized anti-11-dehydrothromboxane B2 antibody.
